(19) United States
(12) Patent Application Publication
(10) Pub. No.: US 2024/0247223 A1
(43) Pub. Date: Jul. 25, 2024

(54) EXAMINATION METHOD OF A MEASUREMENT TARGET BY LIGHT EMITTED IN A REACTION FIELD

(71) Applicant: KABUSHIKI KAISHA TOSHIBA, Tokyo (JP)

(72) Inventors: Saeko SARUWATARI, Kawasaki (JP); Yoko TOKUNO, Yokohama (JP); Ikuo UEMATSU, Yokohama (JP); Mitsuko ISHIHARA, Tokyo (JP); Shigehisa KAWATA, Niiza (JP); Eiichi AKAHOSHI, Tokyo (JP); Takaaki WADA, Tokyo (JP)

(73) Assignee: KABUSHIKI KAISHA TOSHIBA, Tokyo (JP)

(21) Appl. No.: 18/582,858

(22) Filed: Feb. 21, 2024

Related U.S. Application Data
(63) Continuation of application No. 17/124,789, filed on Dec. 17, 2020, now abandoned.
Application No. 17/124,789 is a continuation of application No. PCT/JP2019/024605, filed on Jun. 20, 2019.

(30) Foreign Application Priority Data
Jun. 20, 2018 (JP) 2018-117193

(51) Int. Cl. C12M 1/34; C12M 1/00; C12M 1/12; C12Q 1/6897; D01D 5/00; G01N 21/64

(52) U.S. Cl. CPC C12M 41/46; C12M 23/02; C12M 23/22; C12M 25/14; C12Q 1/6897; D01D 5/0007; G01N 21/6428

(57) ABSTRACT

Provided is an examination method wherein a measurement target is produced in a reaction field within a container and reacted with a reagent to cause light emission which is received, and the optical characteristics thereof detected by a detection unit. The reaction field contains a plurality of pieces in a solution in which the reagent dissolves, the pieces being capable of adsorbing the reagent and gradually releasing the adsorbed reagent for reaction with the measurement target. The container is arranged on the detection unit such that the detection unit faces the reaction field.

### CROSS-REFERENCE TO RELATED APPLICATIONS

This application is a continuation of and claims benefit under 35 U.S.C. § 120 to U.S. application Ser. No. 17/124,789, filed Dec. 17, 2020, which is a continuation of and claims benefit under 35 U.S.C. § 120 to PCT Application No. PCT/JP2019/024605, filed Jun. 20, 2019, and claims the benefit of priority under 35 U.S.C. § 119 from Japanese Patent Application No. 2018-117193, filed Jun. 20, 2018, the entire contents of each of which are incorporated herein by reference.

### FIELD

Embodiments described herein relate generally to an examination device, a container used with the examination device, and a manufacturing method of the container used with the examination device.

### BACKGROUND

As personalized medicines for cancers and molecularly targeted therapy have become widely available, the importance of pathological examination for the purpose of determining cell characteristics in more detail is increasing. Since a pathological examination is often treated as a definitive diagnosis, there exists a demand for improvements in diagnostic accuracy and, in turn, accuracy in determining treatment policy.

A conventional pathological diagnosis is conducted through fixation of specimen cells removed from a patient, and visual examination of those fixed (dead) cells for cell characteristics determined by dye-affinity and antibody reactivity, karyotype, and cellular morphology; however, it has been highlighted that such diagnoses tend to be greatly dependent on the techniques and experience of those conducting the examination.

In recent years, molecular pathological examination procedures, such as FACS, FISH, and PCR, have been developed as auxiliary procedures; however, since a target cell content in the specimen is indefinite, those procedures see decision turnovers occur at a certain rate due to oversights or borderline cases.

### BRIEF DESCRIPTION OF THE DRAWINGS

FIG. 1 is a schematic view of an examination device according to an embodiment.

FIG. 2 is a diagram showing a manufacturing method of a container used with the examination device according to the embodiment.

FIG. 3 is a diagram explaining a manufacturing method of the examination device and a cell detection method.

FIG. 4 is a schematic diagram explaining an example of a detection using the examination device according to the embodiment.

FIG. 5 is a schematic diagram explaining another example of the detection using the examination device according to the present embodiment.

FIG. 6A is a schematic diagram showing a status of a substance (internal substance) and a carrier before incorporation into a cell in the process of a light-emission reaction occurring in a reaction field of the examination device of the present embodiment.

FIG. 6B is a schematic diagram showing a status of incorporating the substance (internal substance) and the carrier into the cell, subsequent to the status shown in FIG. 6A.

FIG. 6C is a schematic diagram showing a status where the substance (internal substance) is released from the carrier and a reporter molecule is generated, subsequent to the status shown in FIG. 6B.

FIG. 6D is a schematic diagram showing a status where light is emitted as a result of a reaction between the reporter molecule and the substrate, subsequent to the status shown in FIG. 6C.

FIG. 7A is a schematic diagram explaining the behavior of a sheet member when light emission occurs in the reaction field as shown in the example shown in FIG. 6A through FIG. 6D, and explaining the adsorption of a part of a substrate by the sheet member.

FIG. 7B is a schematic diagram explaining gradual release of the substrate adsorbed by the sheet member as shown in FIG. 7A to the reaction field.

FIG. 8 is a chart showing an example of a change with time in light emission intensity detected by the detection unit when the light emission occurs in the reaction field as shown in the example of FIG. 6A to FIG. 6D.

FIG. 9A is a schematic diagram showing a status of several types of substances (internal substances) and a carrier before incorporation into a cell in a process of detection of light passing through the reaction field of the examination device of the present embodiment.

FIG. 9B is a schematic diagram showing a status where the multiple types of substances (internal substances) and the carrier are incorporated into the cell, subsequent to the status shown in FIG. 9A.

FIG. 9C is a schematic diagram showing a status where multiple types of molecule are produced in the cell, subsequent to the status shown in FIG. 9B.

FIG. 10 is a schematic diagram of an examination device of a modification of the embodiment.

FIG. 11 is a chart showing results of cell intake rates in the culture using the examination device according to the present embodiment.

FIG. 12A is a bright field image of cells in an observation using a cell detection method according to the present embodiment.

FIG. 12B is an image of light-emitting cells in which a specific gene is expressed in an observation using the cell detection method according to the present embodiment.

FIG. 13A is a schematic diagram showing a solution under a condition X1 that was dropped onto the reaction field in a light emission reaction observed using the examination device according to the present embodiment.

FIG. 13B is a schematic diagram showing a solution under a condition X2 that was dropped onto the reaction field in a light emission reaction observed using the examination device according to the present embodiment.

FIG. 13C is a schematic diagram showing a solution under a condition X3 that was dropped onto the reaction field in a light emission reaction observed using the examination device according to the present embodiment.

FIG. 14 is a chart showing a change with time in intensity of detected emitted light in a light emission reaction observed using the examination device according to the present embodiment.

### DETAILED DESCRIPTION

According to one embodiment, an examination device includes a detection unit, a container placed above the detection unit and made of a material having light-emitting properties, and a sheet member placed in the container.

According to one embodiment, a manufacturing method of above-described examination device is provided. In the manufacturing method, the sheet member is directly formed in the container by the electrospinning method.

According to one embodiment, a cell detection method which uses above-described examination device is provided. In the cell detection method, a group of specimen cells are cultured in the container, and a reagent capable of visualizing characteristics of the group of specimen cells as optical characteristics is brought into contact with the group of specimen cells. In the cell detection method, the optical characteristics are obtained by the detection unit, and target cells included in the group of specimen cells are distinguished based on the optical characteristics.

According to one embodiment, an examination device container used with above-described examination device is provided.

According to one embodiment, manufacturing method of above-described examination device container is provided. In the manufacturing method, the sheet member is directly formed in the container by the electrospinning method.

According to one embodiment, an examination device includes a reagent, a sheet and a detection unit. The reagent reacts with a measurement target and thereby causes light emission. The sheet is capable of adsorbing the reagent and gradually releasing the adsorbed reagent. The detection unit detects optical characteristics of the light emission caused by the reaction between the measurement target and the reagent.

According to one embodiment, in an examination method, light emission is caused by a reaction between a reagent and a measurement target in a reaction field in which a sheet capable of adsorbing and gradually releasing the reagent is placed. In this examination method, a detection unit arranged near the reaction field receives light emitted in the reaction field, and optical characteristics of the received light emitted in the reaction field is detected.

### Embodiment

### <Examination Device>

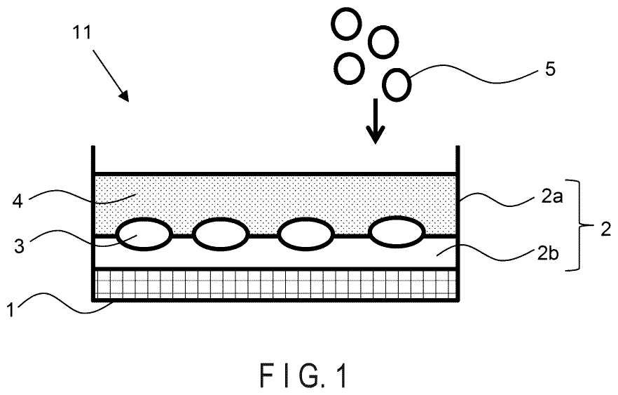

The examination device 11 of the present embodiment shown in FIG. 1 includes a detection unit 1 and a container 2 arranged above the detection unit 1.

### [Container]

The container 2 includes a case 2a and a sheet member 2b stored in the case 2a. The sheet member 2b functions as a platform where cells are cultured. The detection unit 1 and the sheet member 2b face each other, with the case 2a being partially interposed therebetween.

### (Case)

The case 2a stores the sheet member 2b. The case 2a is a container for culturing cells 3 on or within the sheet member 2b accommodated in the case 2a, and for detecting cultured cells 3. For this reason, it is preferable that the case 2a is made of a material that neither influences nor is influenced by the sheet member 2b, the cells 3, a culture solution 4 for culturing the cells 3, a reagent 5 added to detect cells, and the like. Furthermore, it is preferable that the case 2a is made of a material that transmits light of a wavelength necessary to detect cells. Specifically, the material may be quartz glass, polystyrene, polypropylene, polyethylene terephthalate, ABS resin, polyvinyl chloride resin, polycarbonate, polymethylpentene, polytetrafuloroethylene, 4-fluorine fluoride resin, PTFE resin, PFA, acrylic resin, unsaturated polyester resin, epoxy resin, melamine resin, phenol resin, urethane resin, polyethersulfon, permanox, etc. Although not shown, the case 2a may be designed to accommodate the attachment of a lid thereto, so that influences of an environment outside of the case 2a, such as outside air or light, can be shut out.

### (Sheet Member)

For the sheet member 2b, a material suitable for culturing cells 3 thereon is selected. Specifically, a resin on which an irregular surface is formed by a nanoimprint technique, a resin on which sheet-shaped fiber is formed, or the like, can be used. Particularly, a sheet-shaped sheet member 2b made of fibers having an average diameter of 10 μm or smaller is preferable. In this case, it is preferable that the fibers making up the sheet member 2b are randomly oriented. Although the reasons are unknown, it is assumed that random orientations would yield an uneven surface where cells can be easily adhered and grow without being restricted to certain directions, and such a surface is capable of culturing a great variety of cells. The sheet member 2b can be manufactured by any known method, preferably an electrospinning method. The sheet member 2b manufactured by an electrospinning method would be a porous sheet with a flocculent texture. The sheet manufacturing method by an electrospinning method is as follows.

A surface shape of the sheet member 2b may be a square, a rectangular, a rhombus, a circle, or a hexagon, etc. In order to efficiently culture and detect a small amount of cells 3 included in trace amounts of samples, it is preferable if an area of the sheet member accommodated in the case 2a is small. Specifically, a preferable width of the sheet member 2b is 90 mm or less, more preferably 30 mm or less, much more preferably 5 mm or less. If the width is 30 mm or less, it is possible to culture a sufficient number of cells above a light-receiving unit, while the culture environment is suitably maintained. The width of the sheet member is determined by a minimum value of a distance measured from one edge to the other edge in parallel lines in a three-dimensional image of the sheet member, which is observed from a thickness direction using, for example, a digital microscope manufactured by Keyence Corporation, and subjected to an image analysis for distance measurement. A lens and an observation magnification rate that allow observation of an entire sheet member are selected, and if necessary, an image joining function using an XY stage may be used. As a digital microscope, VHX-6000 manufactured by Keyence Corporation may be used.

A lower height (smaller thickness) of the sheet member 2b for detecting cells 3 is more preferable. Specifically, 150 μm or smaller is a preferable thickness of the sheet member 2b. The thickness of 100 μm or smaller is more preferable, and the thickness of 30 μm or smaller is much more preferable. The thickness is 100 μm or smaller helps realize a clear observation even in a situation where the sensitivity of a receiving unit sensor is extremely low or an amount of light emitted from cells 3 is insufficient. The thickness of the sheet member 2b is calculated by a measuring method selected in accordance with the material and shape of the sheet member, such as a non-contact laser displacement gauge, a contact film thickness meter digimatic indicator, a three-dimensional shape measuring machine digital microscope, and a scanning electron microscope observation of an ion milling-processed cross section after resin embedding.

<Manufacture of Sheet with Electrospinning Method>

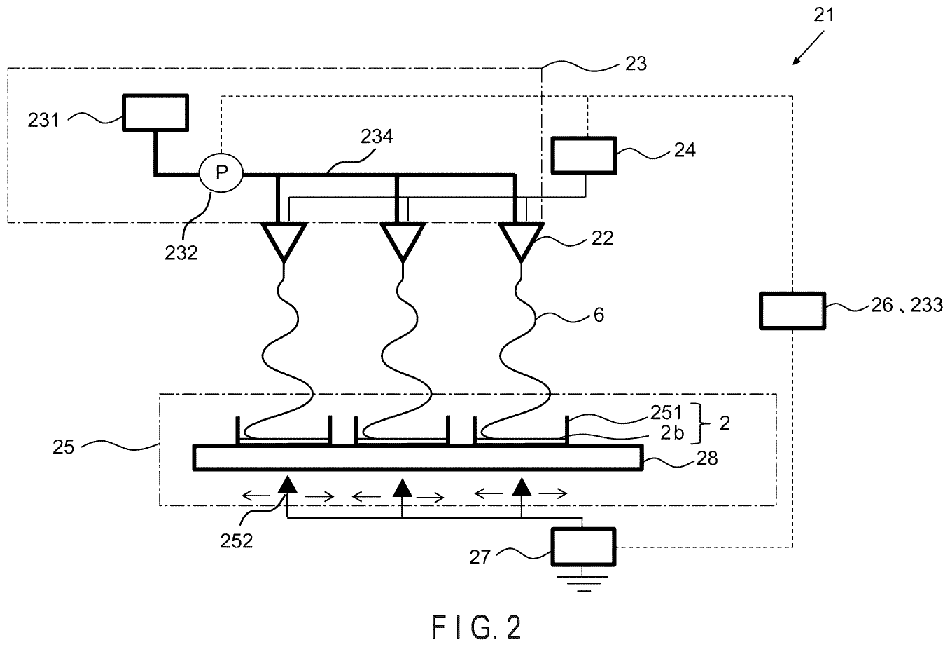

FIG. 2 is a schematic drawing of the electrospinning apparatus 21 when the sheet member 2b is manufactured using an electrospinning method. As shown in FIG. 2, the electrospinning apparatus 21 includes a plurality of nozzles 22, a raw material liquid supply unit 23, a power supply 24, a collecting unit 25, and a control unit 26.

### (Nozzle)

Each nozzle 22 is pin-shaped. In the inside of the nozzle 22, a hole for discharging a raw material liquid is provided. The nozzle 22 is made of an electrically conductive material. Preferably, the material of the nozzle 22 has electrical conductivity and resistance to a raw material liquid. The nozzle may be made of stainless steel, for example.

### (Raw Material Supply Unit)

The raw material liquid supply unit 23 has a storage unit 231, a supply unit 232, a raw material liquid control unit 233, and piping 234.

### (Storage Unit)

A storage unit 231 stores a raw material liquid. The storage unit 231 is made of a material having resistance to a raw material liquid. The storage unit 231 may be made of stainless steel, for example.

The raw material liquid is a high-polymer material, which is made into the fibers 6, dissolved in a solvent. The high-polymer material may be a biocompatible material selected from industrial materials and tissue-derived biomaterial, for example. Examples of the industrial materials are: polypropylene, polyethylene, polystyrene, polyethylene terephthalate, polyvinyl chloride, polycarbonate, nylon, aramid, polyacrylate, polymethacrylate, polyimide, polyamide-imide, polyvinylidene fluoride, polyether sulfone, polyurethane, etc. Examples of tissue-derived biomaterial are: collagen, proteoglycan, chondroitin sulfate proteoglycan, heparin sulfate proteoglycan, keratan sulfate proteoglycan, dermatan sulfate proteoglycan, hyaluronic acid, glycosaminoglycan, fibronectin, laminin, tenascin, entactin, elastin, fibrin, and gelatin. Among all, collagen has high biocompatibility, and exhibits properties suitable for culturing the cells 3. In addition, if the sheet member 2 is made of highly hydrophilic collagen, a refractive index difference between the sheet member 2b in contact with the culture solution 4 and water is small, and high transparency can thereby be obtained. The high-polymer material is not limited to the given examples.

Any solvent can be used, provided that a high-polymer material can dissolve into the solvent. The solvent can be changed as appropriate in accordance with a high-polymer material to dissolve. The solvent may be, for example, water, acetic acid, hydrochloric acid, methanol, ethanol, isopropyl alcohol, n-buthanol, trifluoroethanol, hexafuloro-2-propanol, trifluoro acetic acid, acetone, benzene, toluene, acetonitryl, tetrahydrofuran, dichloromethane, diethyl ether, acetic acid ethyl, cyclohexanone, N,N-dimethylformamide, N,N-dimethylacetamide, N-methyl-2-pyrrolidone, or dimethylsulfoxide. The high-polymer material and the solvent are not limited to the given examples.

### (Supply Unit)

The supply unit 232 supplies a raw material liquid stored in the storage unit 231 to the nozzles 22. The supply unit 232 may be a pump having resistance to a raw material liquid, for example.

### (Raw Material Liquid Control Unit)

The raw material liquid control unit 233 controls a flow amount and a pressure of a raw material liquid supplied to the nozzles 22, so that the raw material liquid inside the nozzles 22 will not be pushed out from the discharging port when a new raw material liquid is supplied to the inside of the nozzles 22. An amount of control in the raw material liquid control unit 233 can be changed as appropriate based on a dimension of the discharging port or viscosity of the raw material liquid. The amount of control in the raw material liquid control unit 233 can be calculated by experiment or simulation. Furthermore, the raw material liquid control unit 233 can be configured to switch between the commencement and cessation of supplying a raw material liquid. The raw material liquid control unit 233 can be included as a part of a control unit 26, which will be described later.

### (Piping)

The piping 234 is provided between the storage unit 231 and the supply unit 232, and between the supply unit 232 and the nozzles 22. The piping 234 serves as a flow path of a raw material liquid. The piping 234 is made of a material having resistance to a raw material liquid.

### (First Power Supply)

The first power supply 24 applies a voltage so as to produce a relative potential difference between each nozzle 22 and the collecting unit 25. The polarity of a voltage (driving voltage) applied to the nozzle 22 is either positive or negative. If a negative voltage is applied to the nozzle 22, however, electrons are released from the end of the nozzle 22 and this tends to cause irregular electric discharge. For this reason, the polarity of a voltage applied to the nozzle 22 should preferably be positive.

The voltage applied to the nozzle 22 may be changed as appropriate in accordance with a type of the high-polymer material included in the raw material liquid or a distance between the nozzle 22 and the collecting unit 25. For example, the first power supply 24 can apply a voltage to the nozzle 22 so as to render a potential difference between the nozzle 22 and the collecting unit 25 10 kV or more. In this case, if the nozzle is plate-shaped, the voltage applied to the nozzle is around 70 kV. On the other hand, if the nozzle is pin-shaped according to the present embodiment, the voltage applied to the nozzle 22 is 50 kV or lower. Reduction of a driving voltage can be thus achieved.

The first power supply 24 may be a direct-current high-voltage power supply, for example. The first power supply 24 may output a direct-current voltage between 10 kV and 100 kV, for example.

### (Collecting Unit)

The collecting unit 25 includes a collecting body 251, an accumulation adjusting unit 252, and a second power supply 27.

### (Collecting Body)

The collecting body 251 is provided on a side where a raw material liquid is discharged, and faces the nozzles 22. In the present embodiment, the above-described case 2a can be used as the collecting body 251. By directly accumulating fibers 6 on the case 2a, it is possible to reduce contamination that can affect cells. In the present embodiment, the collecting body 251 is placed on the stage 28.

As another method, a sheet member 2b is separately formed and die-cut to fit the shape of the case 2a. This method is preferable from the viewpoint of productivity in cases where a great variety of sizes and shapes exist for the case member 2a.

Either the direct accumulation of the fibers 6 on the case 2a, or the forming and die-cutting of the sheet member 2b to fit the case 2a can be selected as appropriate in accordance with a usage and purpose.

### (Accumulation Adjusting Unit)

The accumulation adjusting unit 252 faces the nozzles 22, with the collecting body 251 interposed therebetween. The accumulation adjusting unit 252 is made of an electrically conductive material. The accumulation adjusting unit 252 may be made of a metal such as stainless steel, for example. The end of the accumulation adjusting unit 252 on the collecting body 251 side is pointed. The pointy end of the accumulation adjusting unit 252 on the collecting body 251 side induces electric field concentration. This facilitates the production of an electric field between the nozzles 22 and the accumulation adjusting unit 252.

### (Second Power Supply)

The second power supply 27 applies a voltage to the accumulation adjusting unit 252. The second power supply 27 applies a voltage of a polarity reversed to the voltage applied to the nozzle 22 to the accumulation adjusting unit 252. The second power supply 27 may be a direct-current high-voltage power supply, for example. The second power supply 27 may be configured to output a direct-current voltage between 10 kV and 100 kV, for example.

If a voltage of a polarity reversed to the voltage applied to the nozzle 22 is applied to the accumulation adjusting unit 252, an electric field is also produced between the nozzle and the accumulation adjusting unit 252. The electric field produced between the nozzle 22 and the collecting body 251 is changed by the influence of the electric field produced between the nozzle 22 and the accumulation adjusting unit 252. The raw material liquid in the vicinity of the discharging port of the nozzle 22 is drawn out by static electric power acting along an electric line of force. For this reason, if the electric field produced between the nozzle 22 and the collecting body 251 is changed, it is possible to change an area on which the fiber 6 is accumulated. In other words, the accumulation adjusting unit 252 changes the electric field produced between the nozzle 22 and the collecting body 251 so as to change an area on which the fibers 6 are accumulated.

If the accumulation adjusting unit 252 and the second power supply 27 are provided, it is possible to accumulate the fibers 6 in a desired area. Furthermore, if the accumulation adjusting unit 252 and the second power supply 27 are provided, it is possible to ensure uniformity of the thickness of the sheet member 2b, locally accumulate the fibers 6, repair an opening such as a pinhole formed in the sheet member 2b, and control orientations of the fibers 6, for example.

By controlling a voltage applied to the accumulation adjusting unit 252, the electric field produced between the nozzle 22 and the accumulation adjusting unit 252 and, in turn, the electric field produced between the nozzle 22 and the collecting body 251 can be controlled.

A driving apparatus that moves the accumulation adjusting unit 252 may be provided. If the accumulation adjusting unit 252 is moved, it becomes easier to control the electric field. A single power supply can be used as both the first power supply 24 and the second power supply 27.

After the accumulation of the fibers 6 is completed, since the power supply is grounded, electrons are supplied to the accumulation adjusting unit 252 through this grounding, and natural discharge can also be de-electrified. If an amount of electrification is large, an electrification discharging method using a contact to a conductor may also be adopted.

### (Controller Unit)

The control unit 26 controls the operations of the supply unit 232, the raw material liquid control unit 233, the first power supply 24, and the second power supply 27. The control unit 26 may be a computer having a CPU (central processing unit) and a memory, for example.

### <Operation of Electrospinning Apparatus>

Next, the operation of the electrospinning apparatus 21 is described. The raw material liquid remains in the vicinity of the discharging port of the nozzle 22 due to surface tension.

The first power supply 24 applies a voltage to each nozzle 22. Then, the raw material liquid in the vicinity of the discharging port of the nozzle 22 is electrified in a predetermined polarity.

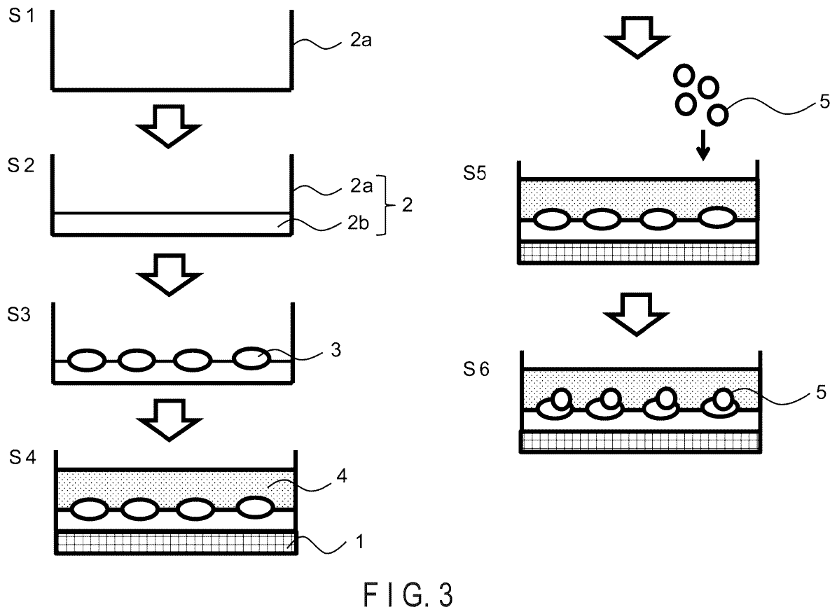

An electric field is produced between the nozzle 22 and the collecting body 251. Then, when static electric power acting along the electric line of force becomes relatively larger than the surface tension of the liquid, the raw material liquid in the vicinity of the discharging port of the nozzle 22 is drawn out toward the collecting body 251. The drawn raw material liquid is extended, and as the solvent contained in the raw material liquid volatilizes, the fibers 6 are formed. The fibers 6 are accumulated on the collecting body 251 and the sheet member 2b is thereby formed (S2 in FIG. 3). By controlling at least one of the voltage applied to the accumulation adjusting unit 252 or the relative position relationship of the accumulation adjusting unit 252 with respect to the collecting body 251, the area on which the fibers 6 are accumulated can be changed.

### (Sheet Member)

By controlling at least one of the voltage applied to the nozzle 22, the speed of supplying the raw material liquid to the nozzle 22, the type and concentration of the high polymer contained in the raw material liquid, the type of the solvent, or the distance between the nozzle 22 and the collecting body 251, it is possible to bring the average diameter of the fibers 6 constituting the sheet member 2b to the range from 0.05 μm to 10 μm. The average diameter of the fibers 6 contained in the sheet member 2b can be calculated by, for example, averaging the diameters of randomly picked 100 fibers 6 observed in an electron micrograph of the surface of the sheet member 2b.

Furthermore, by suppressing volatilization of the solvent contained in the raw material liquid drawn out of the nozzle 22, it is possible to allow the sheet member 2b to contain thick fibers 6. It is thereby possible to help the fibers 6 adhere to each other and to improve adhesion between the fibers 6. If the adhesion between the fibers 6 is improved, it is possible to suppress an increase in thickness that occurs in a case where the sheet member contains a culture solution. Thus, it becomes possible to clearly observe cells in cases where the sensor sensitivity of light-receiving unit is extremely low, or where an amount of light emitted from cells is insufficient, for example. Furthermore, it is possible to make the shape of the thick fibers in a flat-ribbon shape, pleats, branches, beads, and the like. It is thereby possible to effectively obtain an effect on plane-direction adhesion of the fibers contained in the sheet member, to suppress an excessive increase in thickness of the sheet member, or to provide appropriate space to the sheet member. The width (or fiber diameter in some cases) of the thick fibers 6 may fall within the range from 6 μm to 20 μm, for example. The presence rate of the thick fibers 6 contained in the sheet member 2b can be calculated by, for example, dividing the number of fibers 6 having the width of 6 μm or thicker among some randomly picked 100 fibers 6 observed in an electron micrograph of the surface of the sheet member 2b (for example, a scanning electron micrograph) by the total number of the fibers. The ratio of the thick fibers 6 is preferably in the range from 1% to 70%. It is more preferable if the ratio falls within the range from 5% to 60%. If the ratio is lower than 1%, the effect on adhesion between the fibers 6 cannot be sufficiently obtained. If the ratio is 70% or higher, it is difficult to provide sufficient space to the sheet member. To provide sufficient space to the sheet member, it is more desirable if the ratio of the fibers having the thickness in the range from 6 μm to 20 μm falls within the range from 1% to 70%. The much more preferable range is from 5% to 60%. The volatilization of the solvent from the raw material liquid can be suppressed by adjusting the type of the solvent and the concentration of the high polymer contained in the raw material liquid.

Herein, the details of the method of measuring the width of the fibers are described. The surface of the sheet member is observed using, for example, a digital microscope manufactured by the Keyence Corporation, and a three-dimensional image of the surface is obtained. Next, the fiber length direction is determined for each fiber. An average value of distances from one end of a fiber to the other measured in parallel lines, which are perpendicular to the direction of the fiber length direction, is calculated, and this value is defined as a width perpendicular to the direction of fiber length. A lens and an observation magnification rate that allow observation of an entire of the fiber are selected, and if necessary, an image joining function utilizing an XY stage may be used. As a digital microscope, VHX-6000 manufactured by Keyence Corporation may be used.

By controlling at least one of the voltage applied to the nozzle 22, the speed of supplying the raw material liquid to the nozzle 22, the type and concentration of the high polymer contained in the raw material liquid, the type of the solvent, or the distance between the nozzle 22 and the collecting body 251, it is possible to bring the surface roughness of the sheet member 2b to the range of the arithmetic average height, 0.1 μm≤Sa≤5 μm, and the range of the maximum height, 1 μm≤Sz≤90 μm. Herein, the arithmetic average height Sa represents an average of absolute values of the differences between the height at respective points and the height of an average plane of the surface. A maximum height Sz represents a distance from a highest point to a lowest point of the surface. Since the sheet member 2b has surface roughness in the order of microns, it is possible to provide a surface structure having unevenness that allows for easy adhesion of cells. The surface roughness of the sheet member 2b is observed using the Keyence digital microscope, for example, and three-dimensional images of five randomly selected spots are obtained. Herein, suppose the measurement magnification is set to ×1000, and an observation range per spot is 0.084 mm². Image analysis is performed on the three-dimensional image to calculate the arithmetic average height Sa and the maximum height Sz. As a digital microscope, VHX-6000 manufactured by Keyence Corporation may be used.

By suppressing volatilization of the solvent included in the raw material liquid drawn out of the nozzle 22, it is possible to bind a portion of the fibers 6 included in the sheet member 2b to the detection unit 1. With the binding sites provided, it is possible to prevent peeling of the sheet member 2b from the detection unit 1. As a method of checking binding sites, the surface of the detection unit 1 is observed after the sheet member 2b is peeled off by, for example, an adhesive tape. As an adhesive tape, a paper adhesive tape with an acrylic adhesive can be used, for example.

### <Detection Unit>

The cells 3 are placed on the sheet member 2b in the container 2 manufactured as described above (S3 in FIG. 3), and the cells 3 are soaked in a culture solution and cultured under conditions such as a predetermined temperature and a period of time (S4 in FIG. 3). Directly placing the container in which cells are thus cultured on the detection unit 1 allows a direct detection of the status of the cells through the bottom surface of the case 2a of the container 2.

The detection unit includes a group of lenses and a light-receiving unit. The group of lenses has a role of guiding light passing through the container to the light-receiving unit. The group of lenses may be of focal or non-focal type, and can be selected in accordance with purpose of use. A microlens array is an example configuration for the group of lenses.

The light-receiving unit is a sensor capable of receiving light passing through the group of lenses. An example of the light-receiving unit is a CMOS sensor.

### <Cell Detection Method>

Although it is possible to observe the cells 3 as an examination target by placing the container 2 in which the cells 3 are cultured on the detection unit 1 as described above, for better observation, a reagent 5 that exhibits a specific reaction with the cultured cells 3 may be dropped onto the cells (S5 in FIG. 3). This makes it possible to perform more accurate observations suitable for purpose of use. For example, a reagent used for distinguishing living cells from dead cells may be dropped onto the cultured cells 3, or a reporter vector DNA that includes a light-emitting enzyme, such as luciferase, for visualizing an expression of a specific gene may be introduced and a light-emitting substrate may be dropped so as to improve the capability of distinguishing cells having a specific quality (S6 in FIG. 2).

For example, calcein may be added as a reagent to observe living cells energized by light with a wavelength of 490 nm, making it thereby possible to observe light with a wavelength of 515 nm, and improve cell distinction.

### [Example of Reagent]

In one example, the reagent 5 includes a substance that produces a signal in accordance with cell activity. The substance that produces a signal in accordance with the activity of the cell may be an internal substance encapsulated by a carrier. In one example, a component that includes a measurement target is generated by a substance that produces a signal in accordance with activity of a cell. The substance (internal substance) that produces a signal in accordance with the activity of a cell may include at least one of the following: a molecule that recognizes a biomolecule, protein, antibody, enzyme, nucleic acid, vector DNA, plasmid, a stain for protein, or a stain for DNA. The carrier may include at least one of a tissue-derived molecule, a biocompatible molecule, a biolysis molecule, a lipid molecule, or a polymer, and liposome is a specific example of the carrier. The reagent 5 may include a substrate (luminescent substrate) that causes light emission upon a reaction with a component that includes a measurement target generated in the cell.

### [Specific Example of Detection]

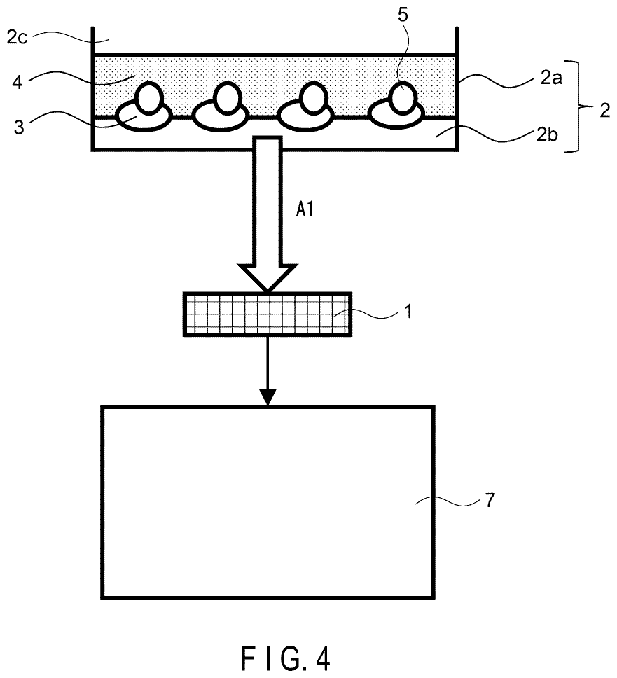

In the following, a specific example of the detection using the above-described examination device will be explained. FIG. 4 shows an example of the detection. In the example shown in FIG. 4, a reaction field 2c is formed in the case 2a of the container 2, and the sheet member (sheet) 2b is placed on the bottom surface of the case 2a in the reaction field 2c. Then, the cells 3 are placed on the sheet member 2b, and then soaked in a culture solution 4 in the reaction field 2c. By dropping the reagent 5 onto the reaction field 2c, a component as a measurement target is generated in the cell 3 in accordance with a substance that generates a signal in accordance with the activity of the cell 3, and the reaction, etc. between the generated component and the component included in the reagent 5 causes a light emission reaction in the reaction field 2c. Then, the detection unit 1 receives light emitted in the reaction field 2c (the arrow A1). In other words, the light emitted in the reaction field 2c passes through the sheet member 2b, and is guided to the detection unit 1. In one example, the detection unit 1 includes a spectrophotometer such as a plate reader, and detects photon quantities received during a predetermined period of time. The detection unit 1 thereby detects light emission intensity (an amount of light emission) in the reaction field 2c.

In the case 2a of the example shown in FIG. 4, at least the part arranged between the reaction field 2c and the detection unit 1 is made of a material having light-transmitting properties. In the example of FIG. 4, a processing apparatus 7 having a processor and a storage medium, etc. is provided in the examination device. The processor of the processing apparatus 7 includes a CPU (central processing unit), an ASIC (application specific integrated circuit), or an FPGA (field programmable gate array), etc. In the example shown in FIG. 4, the processing apparatus 7 obtains a detection result at the detection unit 1. Then, the processing apparatus 7 determines light emission intensity in the reaction field 2c based on the obtained detection result, or notifies an examiner, etc. about the obtained detection result through image displaying, etc.

In another example, the detection unit 1 includes a CMOS sensor or a camera, etc., and obtains an image of the reaction field 2c in a state of light emission as described above. In other words, the image of the reaction field 2c is detected by the detection unit 1 as optical characteristics. In this case, the processing apparatus 7 may perform a determination process based on the image of the reaction field 2c obtained by the detection unit 1, or display the image obtained by the detection unit 1 on the screen.

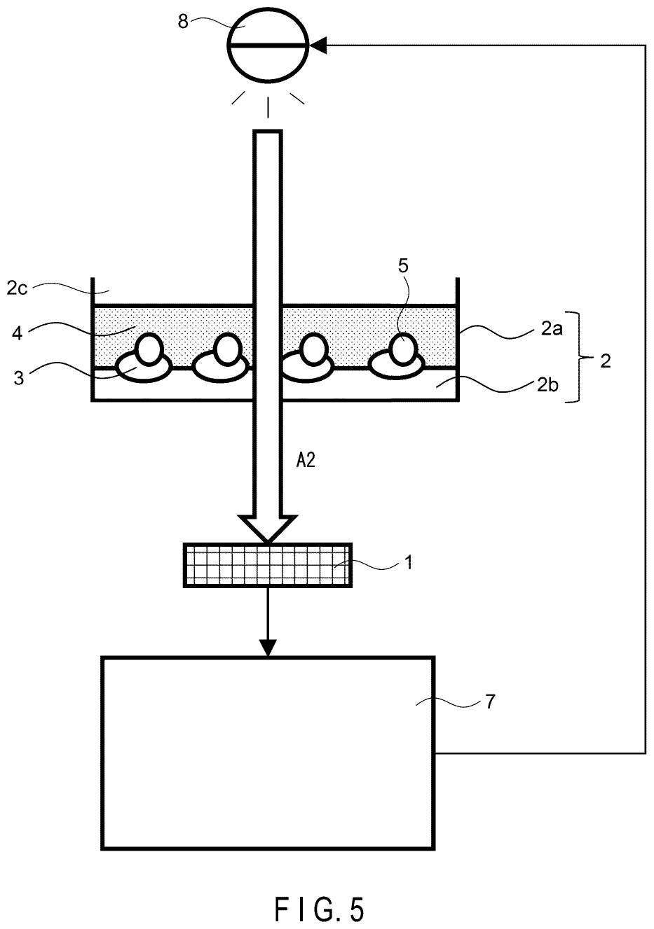

FIG. 5 shows a different example of the detection from that shown in FIG. 4. In the example of FIG. 5, the cells 3 are placed on the sheet member 2b and soaked in the culture solution 4 in the reaction field 2c. Then, by dropping the reagent 5 onto the reaction field 2c, the component, which is a measurement target, is generated in the cells 3. In the example of FIG. 5, a light source 8 is provided, and the reaction field 2c is irradiated by the light source 8. Then, the light irradiating the reaction field 2c passes through the reaction field 2c (the sheet member 2b), and the light passing through the reaction field 2c is received by the detection unit 1 (the arrow A2). In the case 2a in the example shown in FIG. 5, at least the part arranged between the reaction field 2c and the light source 8 and the part arranged between the reaction field 2c and the detection unit 1 are made of a material having light-transmitting properties.

In one example, the wavelength spectrum of the light emitted from the light source 8 changes in the reaction field 2c by the generated component (expressed component). The detection unit 1 then receives the light of which the wavelength spectrum has changed in the reaction field 2c. Then, through the processes in the detection unit 1 and in the processing apparatus 7, an amount of change in the wavelength of the light when passing through the reaction field 2c is detected. In another example, the light irradiated by the light source 8 is attenuated in the reaction field 2c by the generated component. The detection unit 1 then receives the light attenuated in the reaction field 2c. Then, through the processes in the detection unit 1 and in the processing apparatus 7, an amount of attenuation of the light when passing through the reaction field 2c is detected. In other words, an amount of change in the intensity of light when passing through the reaction field 2c is detected. In the example of FIG. 5, the detection unit 1 includes either one of an optical sensor capable of detecting parameters relating to optical characteristics, or an image sensor, such as a CMOS sensor, etc. for obtaining an image of the reaction field.

### [Specific Examples of Detection of Light Emission in Reaction Field]

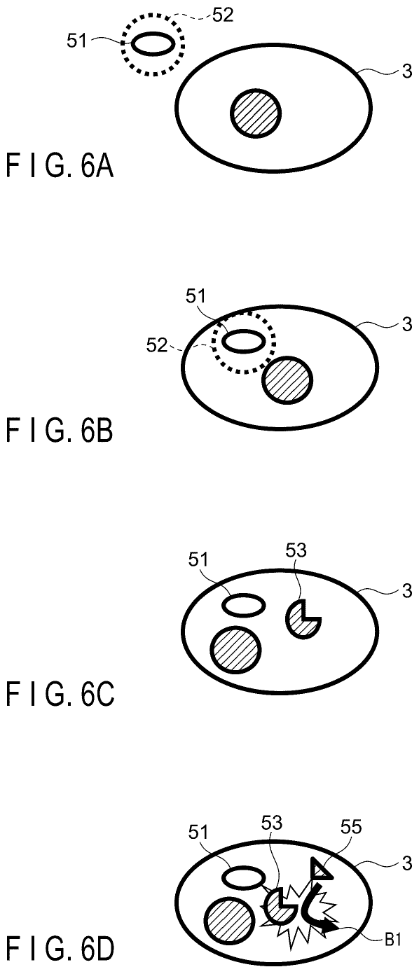

FIGS. 6A through 6D show an example of a light emission reaction in the reaction field 2c. In the example of FIGS. 6A through 6D, the reagent 5 dropped onto the reaction field 2c includes the above-described substance (internal substance) 51 that produces a signal in accordance with the activity of the cell, and the substance 51 is encapsulated by the carrier 52. As shown in FIG. 6A, when the substance 51 and the carrier 52 are charged into the reaction field 2c, they are incorporated into the cells 3, as shown in FIG. 6B. The carrier 52 decomposes after being incorporated into the cell 3. FIG. 6A shows a state before the substance 51 and the carrier 52 are incorporated into the cell 3. In the state shown in FIG. 6B, the cell 3 is placed on the sheet member 2b in the reaction field 2c, and cultured, being soaked in the culture solution 4.

After the substance 51 is incorporated into the cell 3, the reporter molecule 53 is produced in the cell 3 in accordance with activity of the cell 3, as shown in FIG. 6C. In one example, luciferase is expressed as a reporter molecule 53. The reagent 5 includes the above-described substrate (light-emitting substrate) 55. As shown in FIG. 6D, the substrate 55 charged into the reaction field 2c reacts with the reporter molecule 53 produced in the cell 3 (the arrow B1). In the reaction field 2c, a reaction between the reporter molecule 53 and the substrate 55 causes light emission. Then, the detection unit 1 detects light generated as a result of the reaction between the reporter molecule 53 and the substrate 55.

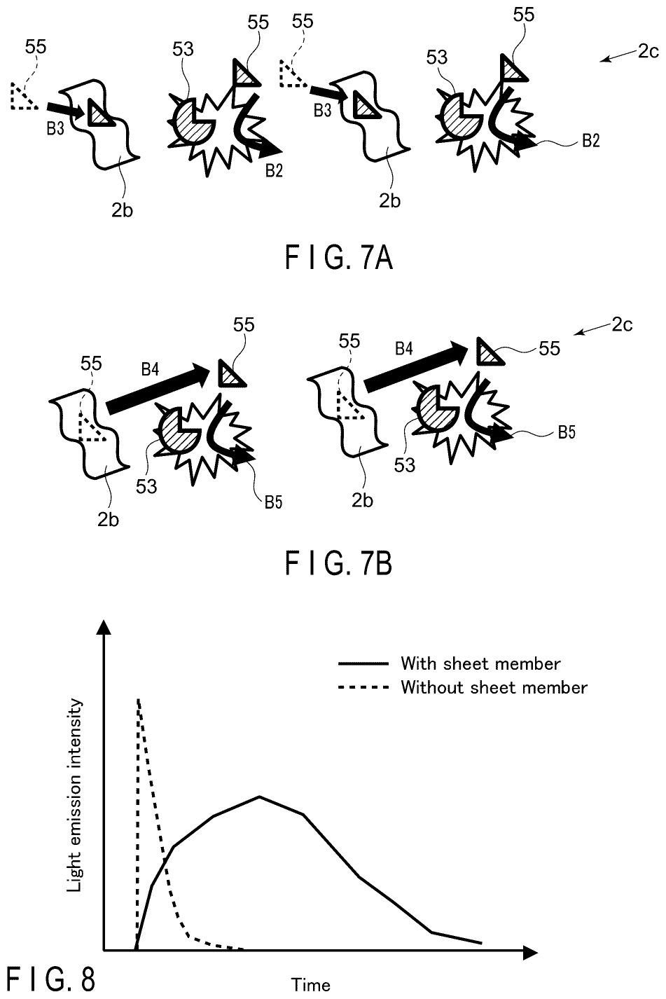

FIGS. 7A and 7B show a behavior of the sheet member 2b in the case where light emission occurs in the reaction field 2c as in the example shown in FIGS. 6A through 6D. As shown in FIG. 7A, when the substrate 55 is charged into the reaction field 2c where the reporter molecule 53 has been produced, a part of the charged substrate 55 reacts with the reporter molecule 53 (the arrow B2). Then, light emission is caused by the reaction between the reporter molecule 53 and the substrate 55. On the other hand, another part of the charged substrate 55 is adsorbed by the sheet member 2b (the arrow B3). Unlike the cells 3 and the reporter molecule 53, the substrate 55 can invade the inside of the sheet member 2b made of the fibers in the above-described manner. For this reason, the substrate 55 adsorbed by the sheet member 2b does not react with the reporter molecule 53. In other words, the sheet member 2b suppresses the reaction between the substrate 55 adsorbed by the sheet member 2b and the reporter molecule 53.

The substrate 55 adsorbed by the sheet member 2b is gradually released to the reaction field 2c as shown in FIG. 7B (the arrow B4). In other words, the substrate 55 adsorbed by the sheet member 2b is gradually released to the reaction field 2c over a long period of time. Then, the substrate 55 released to the reaction field 2c reacts with the reporter molecule 53 (the arrow B5). The light emission is thus caused in the reaction field 2c.

FIG. 8 shows an example of a change with time in light emission intensity detected by the detection unit 1 when the light emission occurs in the reaction field 2c as shown in the example of FIG. 6A to FIG. 6D. In FIG. 8, the abscissa axis represents time, and the ordinate axis represents light emission intensity. In FIG. 8, the dotted line shows the change with time of the light emission intensity in a case where the sheet member 2b is not arranged in the reaction field 2c, and the solid line shows the change with time of the light emission intensity in the case where the sheet member 2b is arranged in the reaction field 2c as in the example of FIGS. 7A and 7B.

If the sheet member 2b is not arranged in the reaction field 2c, the majority part of the charged substrate 55 reacts with the reporter molecule 53 as soon as the substrate 55 is charged into the reaction field 2c, and light emission occurs. Then, when the light emission reaction that occurs immediately after the charging of the substrate 55 stops, the reaction between the substrate 55 and the reporter molecule 53 hardly occurs in the reaction field 2c, and then the light emission hardly occurs. Thus, as shown in FIG. 8, in the case where the sheet member 2b is not arranged in the reaction field 2c, the light emission intensity becomes maximum, namely reaches a peak value, immediately after the substrate 55 is charged into the reaction field 2c. Then, after the light emission intensity reaches its peak value, the light emission intensity rapidly decreases.

On the other hand, in the case where the sheet member 2b is arranged in the reaction field 2c, a part of the charged substrate 55 is adsorbed by the sheet member 2b as described above, and the reaction between the substrate 55 adsorbed by the sheet member 2b and the reporter molecule 53 is suppressed. For this reason, in the case where the sheet member 2b is arranged, the light emission intensity that occurs immediately after the charging of the substrate 55 is lower than in the case where the sheet member 2b is not arranged. Then, in the case where the sheet member 2b is arranged, the peak value (maximum value) of the light emission intensity becomes lower than in the case where the sheet member 2b is not arranged.

However, in the case where the sheet member 2b is arranged, the substrate 55 adsorbed by the sheet member 2b is gradually released to the reaction field 2c, and the substrate 55 released in the reaction field 2c reacts with the reporter molecule 53. For this reason, in the case where the sheet member 2b is arranged, the light emission continues for a longer period of time than in the case where the sheet member 2b is not arranged. Then, in the case where the sheet member 2b is arranged, the light emission intensity gradually decreases even after the intensity reaches its peak value.

If the sheet member 2b is arranged, a part of the substrate 55 is adsorbed by the sheet member 2b as described above. For this reason, in the case where the sheet member 2b is arranged, the concentration of the substrate 55 in the reaction field 2c at the time when light emission occurs is lower than in the case where the sheet member 2b is not arranged. Because of the lower concentration of the substrate 55 in the reaction field 2c, the light emission quantum yield to the substrate 55 is higher in the case where the sheet member 2b is arranged than in the case where it is not arranged. For this reason, in the case where the sheet member 2b is arranged, a probability of light emission per substrate 55 is higher than that in the case where it is not arranged. Because of the higher light emission quantum yield to the substrate 55, a net amount of light emission from the beginning to the end of the light emission is larger in the case where the sheet member 2b is arranged than in the case where it is not arranged.

As described above, the sheet member 2b is capable of adsorbing and gradually releasing the substrate 55. Because of the adsorption of a part of the substrate 55 by the sheet member 2b and the gradual release of the substrate 55 adsorbed by the sheet member 2b, the light emission continues for a longer period of time, and a net amount of light emission becomes larger. Accordingly, with the sheet member 2b arranged in the reaction field 2c, it is possible to receive by the detection unit 1 the light generated in the reaction field 2c for a longer period of time, and in turn, to perform the detection of the optical characteristics by the detection unit 1, etc. for a longer period of time. Furthermore, if the time for receiving the emitted light by the detection unit 1, namely exposure time, is longer, the net amount of light received by the detection unit 1 becomes larger; therefore, the optical characteristics are detected with high sensitivity by the detection unit 1. The high-sensitivity detection by the detection unit 1 improves examination accuracy using the examination device.

In an example, an accumulation value of the light emission intensity in the reaction field 2c during a predetermined accumulation time is detected by the detection unit 1 and the processing apparatus 7. In this case, the detection unit 1 and the processing apparatus 7 may detect an amount of photons received by the detection unit 1 during the predetermined accumulation time as an accumulation value of the light emission intensity. Furthermore, the detection unit 1 and the processing apparatus 7 may detect an amount of photons received by the detection unit 1 at predetermined intervals (for example, every 1 second) in the predetermined accumulation time. In this case, the detection unit 1 and the processing apparatus 7 calculate a sum of the amounts of photons detected at predetermined intervals as an accumulation value of the light emission intensity. In one example, the predetermined accumulation time is any length of time between 3 seconds and 60 minutes.

As described earlier, if the sheet member 2b is arranged in the reaction field 2c, the light emission continues for a longer period of time, and a net amount of light emission is larger. For this reason, the high-sensitivity detection can be achieved by the detection by the detection unit 1, etc. using the accumulation value of the light emission intensity as a parameter relating to the optical characteristics.

### [Specific Examples of Detection of Light Passing Through Reaction Field]

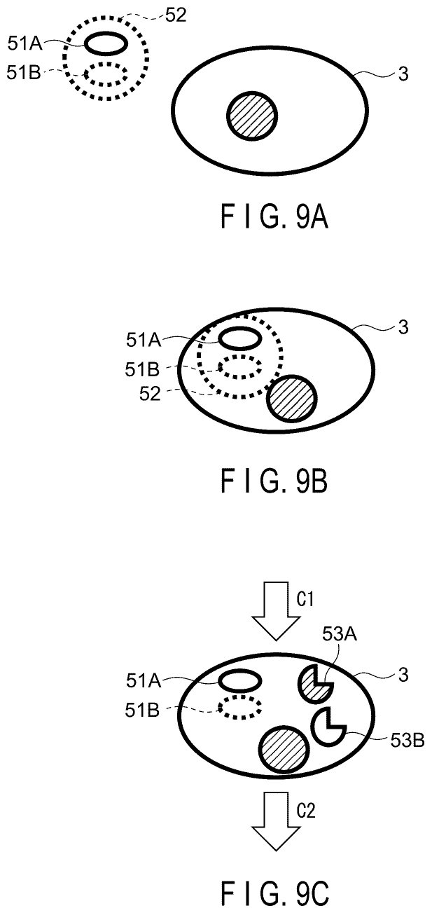

FIGS. 9A through 9C show an example of the detection of light passing through the reaction field 2c. In the example of FIGS. 9A through 9C, the reagent 5 dropped onto the reaction field 2c includes multiple types of the above-described substances (internal substances) 51A and 51B that each produce a signal in accordance with the activity of the cell, and the substances 51A and 51B are encapsulated by the carrier 52. As shown in FIG. 9A, the substances 51A and 51B and the carrier 52 are charged into the reaction field 2c, and the substances 51A and 51B and the carrier 52 are incorporated into the cells 3 as shown in FIG. 9B. The carrier 52 decomposes after being incorporated into the cells 3, similarly to the example shown in FIGS. 6A through 6D. FIG. 9A shows a state before the substances 51A and 51B and the carrier 52 are incorporated into the cell 3.

Through the incorporation of the substance 51A into the cell 3, the reporter molecule 53A is produced in the cell 3, as shown in FIG. 9C. Through the incorporation of the substance 51B into the cell 3, the reporter molecule 53B of a type different from the type of the reporter molecule 53A is produced in the cell 3. Thus, in the example of FIGS. 9A through 9C, multiple types of the reporter molecules 53A and 53B are produced. In one example, different types of fluorescent proteins are expressed as the reporter molecules 53A and 53B.

In the example of FIGS. 9A through 9C, the light source 8 irradiates the reaction field 2c with excitation light (the arrow C1). Through the irradiation of the reporter molecule 53A produced in the cell 3 with the excitation light, fluorescence occurs in the reaction field 2c. As the reporter molecule 53B produced in the cell 3 is irradiated with excitation light, fluorescence of a color (wavelength) different from that of the reporter molecule 53A occurs in the reaction field 2c. In one example, the reporter molecule 53A is a fluorescent protein that produces green fluorescence by excitation light, and the reporter molecule 53B is a fluorescent protein that produces red fluorescence by excitation light. The detection unit 1 receives fluorescence generated by the reporter molecules 53A and 53B (the arrow C2).

As described above, each of the reporter molecules 53A and 53B causes fluorescence upon adsorption of excitation light. Then, from the excitation light with which the reaction field 2c is irradiated, the wavelength of the fluorescent light received by the detection unit 1 changes. In other words, the light with which the reaction field 2c is irradiated changes in its wavelength spectrum when the light passes through the reaction field 2c. The detection unit 1 receives the fluorescence, and detects an amount of change in the wavelength spectrum when the light passes through the reaction field 2c. Then, the detection unit 1 and the processing apparatus 7, etc. detect intensity of fluorescence generated by each of the reporter molecules 53A and 53B based on a detection result, etc. of an amount of change in the wavelength spectrum, and analyze an expression ratio of each of the reporter molecules 53A and 53B of different types in the cell 3, and the ratio between the reporter molecules 53A and 53B of different types in the cell 3, and the like.

### (Modifications of Embodiment)

In the foregoing embodiment, an aspect in which the container 2 and the detection unit are separately provided is described; however, the embodiment is not limited to this aspect. Specifically, the detection unit 1 may be integrated into the bottom surface of the case 2a from the beginning, and the sheet member 2b may then be formed in this case 2a. This modification can be adopted as appropriate in accordance with a detection target, a resolution required for detection, or the like.

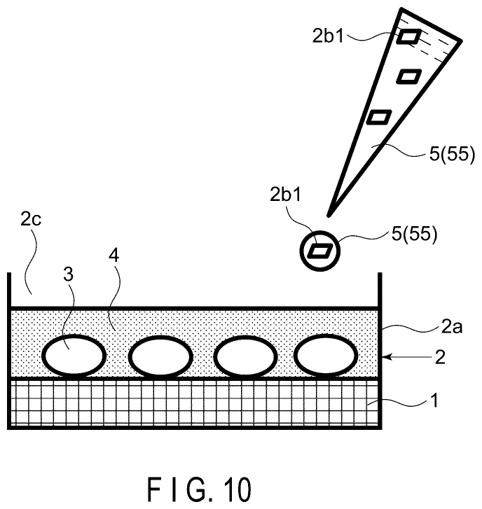

In the foregoing embodiment, etc., the sheet member 2b is spread (placed) on the detection unit 1, for example the bottom surface of the case 2a; however, the embodiment, etc. is not limited to this example. In a modification as shown in FIG. 10, the sheet member 2b is not spread on the bottom surface of the case 2a, but the cells 3 are directly placed on the bottom surface of the case 2a. Even in this modification, the cells 3 are soaked in the culture solution 4 in the reaction field 2c. In this modification, a number of sheet pieces 2b1, which are formed by finely dividing the sheet member 2b, are used, instead of the sheet member 2b. Furthermore, in the present modification, the small sheet pieces 2b1 are dispersed in (stirred into) a solution of the reagent 5 in which the substrate 55, etc. dissolves. Then, the solution of the reagent 5 in which a number of the sheet pieces 2b1 are dispersed is dropped onto the reaction field 2c.

In the present modification, similarly to the example shown in FIGS. 6A through 6D for example, light emission occurs in the reaction field 2c as a result of a reaction between the reporter molecule 53 expressed in the cell 3 and the substrate 55 included in the reagent 5. In the present modification, the sheet pieces 2b1 adsorb a part of the substrate 55 charged into the reaction field 2c, in a manner similar to the adsorption by the sheet member 2b in the foregoing embodiment, etc. The sheet pieces 2b1 then gradually release the adsorbed substrate 55. For this reason, even in this modification, similar to the example shown in FIGS. 7A and 7B for example, the light emission in the reaction field 2c continues for a long period of time, and a net amount of light emission from beginning to end is large.

In the foregoing embodiment, etc., the sheet, such as the sheet member 2b, etc. adsorbs the substrate 55; however, the embodiment, etc., is not limited to this example. In one modification, one of the substance (internal substance) 51 that produces a signal in accordance with activity of a cell, the carrier 52, or the reporter molecule 53 may be adsorbed by the sheet instead of or in addition to the substrate 55. In this case, one of the substance 51, the carrier 52, or the reporter molecule 53, etc. adsorbed by the sheet is to be gradually released.

In the foregoing embodiment, etc., an amount of light emission caused by the reaction of the substance produced in the cell 3, an amount of change in a wavelength spectrum of light by the substance produced in the cell 3, or an amount of attenuation of light by the substance produced in the cell 3 is detected by the detection unit 1, and an examination is performed on the substance produced in the cell 3 as a measurement target; however, the embodiment, etc. is not limited to this example. In other words, an examination device similar to the above-described examination device may be used, tracking a substance other than the substance produced in a cell as a measurement target.

In one modification, an examination is conducted using ATP (adenosine triphosphate) as a measurement target, and quantitative analysis is conducted on the ATP included in a sample. The ATP is a substance used in a reaction elementary process of a biological element that requires energy, and is an index used in a microorganism examination performed on, for example, food. In this modification, the reaction field 2c is formed on a substrate made of a material having light-transmitting properties, and in the reaction field 2c, the sheet member 2b is arranged on the substrate. Then, the detection unit 1 is arranged, relative to the substrate, on the side opposite to the side where the reaction field 2c is formed.

In the examination, luciferin as the substrate (fluorescent substrate 55) and a sample including ATP are dropped onto the reaction field 2c. Then, luciferase is dropped onto the reaction field 2c. Thus, luciferin and ATP reacts with each other with the use of luciferase as an enzyme (catalyst), and light emission occurs in the reaction field 2c. Then, the detection unit 1 receives the light emitted in the reaction field 2c.

In the present modification, the sheet member 2b adsorbs a part of the luciferin (substrate 55) charged into the reaction field 2c. Then, the sheet member 2b gradually releases the adsorbed luciferin. For this reason, even in this modification, similar to the example shown in FIGS. 7A and 7B for example, the light emission in the reaction field 2c continues for a long period of time, and a net amount of light emission from beginning to end is large.

In another modification, using the reaction field 2c and the detection unit 1 similar to those in the modification where the quantitative analysis on ATP is performed, an examination is performed on an oxidation auxiliary material included in a sample as a measurement target, so as to perform quantitative analysis on the oxidation auxiliary material. In one example, the sample is blood, and the oxidation auxiliary material as a measurement target is either a metal ion or an antioxidant organic molecule.

In the examination, luminol as the substrate 55 is dropped onto the reaction field 2c. Then, a reactive oxygen species, such as hydrogen peroxide, and the sample are dropped onto the reaction field 2c. Thus, luminol and the reactive oxygen species react with each other with the use of the oxidation auxiliary material included in the sample as catalyst, and light emission occurs in the reaction field 2c. Then, the detection unit 1 receives the light emitted in the reaction field 2c.

In the present modification, the sheet member 2b adsorbs a part of the luciferin (substrate 55) charged into the reaction field 2c. Then, the sheet member 2b gradually releases the adsorbed luminol. For this reason, even in this modification, similar to the example shown in FIGS. 7A and 7B for example, the light emission in the reaction field 2c continues for a long period of time, and a net amount of light emission from beginning to end is large.

### EXAMPLES

### Example 1

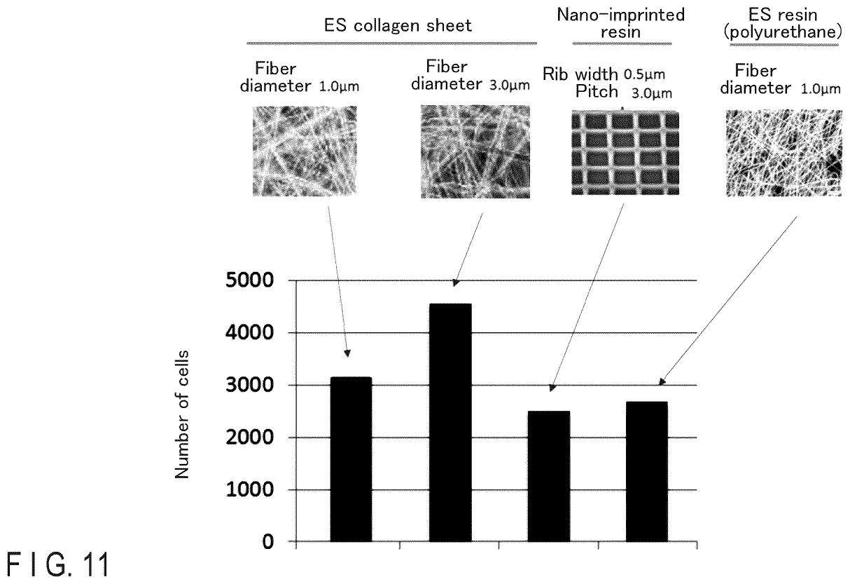

A sheet member 2b was manufactured from each of a nano-imprinted resin, polyurethane, and collagen, and the cell intake ratios of the sheets were observed. The polyurethane sheet and the collagen sheet were manufactured by the above-described electrospinning method, using a glass substrate as a stage. The characteristics of those sheets are shown in Table 1 below, and a result of the cell intake ratio is shown in FIG. 11. The width of each of the sheet members was 18 mm.

TABLE 1

| No. | Material | Structure |
|---|---|---|
| 1 | Collagen | Fiber diameter 1.0 μm |
| 2 | Collagen | Fiber diameter 3.0 μm |
| 3 | Polyurethane | Fiber diameter 1.0 μm |
| 4 | Nano-imprinted resin | Rib width 0.5 μm |
| | | Pitch 3.0 μm |

### Example 2

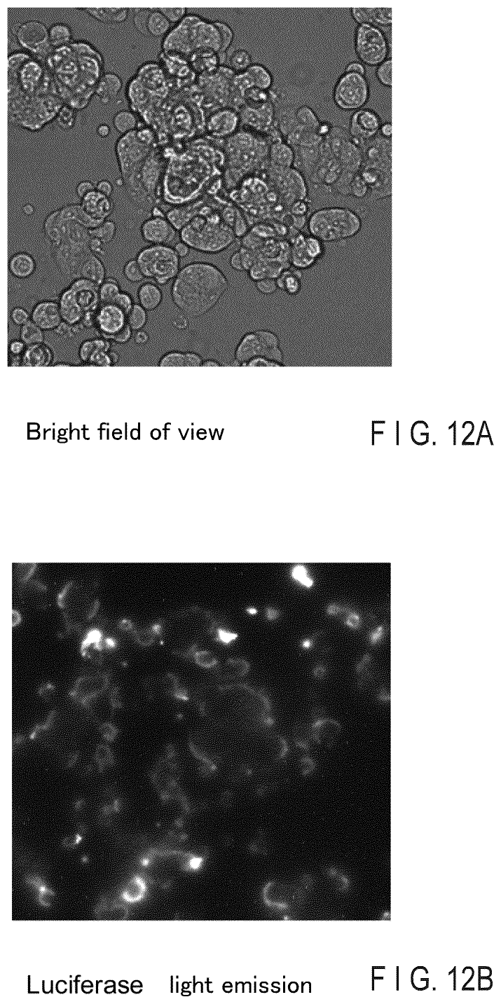

For the purpose of discriminating the cells in which a specific gene is expressed, MCF7 was seeded in a container in which the sheet member No. 2 in the foregoing Example 1 was used, a reporter vector DNA (Promega) obtained by joining a cytomegalovirus promoter to a NanoLuc gene was introduced to the cells, and the cells were cultured for 24 hours, before the container was observed using the examination device. The result is shown in FIGS. 12A and 12B. Although it was possible to equally observe all the cells in a bright field image as shown in FIG. 12A, an image of the fluorescent cells in which a specific gene is expressed was obtained (FIG. 12B). This was due to the gene expression being rendered visualized, and a finding that cells having certain qualities can thereby be easily distinguished.

### Example 3

The sheet member 2b was manufactured using collagen as a material, and the cell intake and performance in discrimination of luminescent cells were evaluated. The sheet member 2b was manufactured using the above-described electrospinning method. The presence ratio of the thick fibers having the width between 6 μm and 20 μm was calculated. MCF7 was seeded in a container in which the sheet members No. 5 to No. 23 were arranged, and a reporter vector DNA (Promega) obtained by joining a cytomegalovirus promoter to a NanoLuc gene was introduced to the cells and cultured for 24 hours, before the container was observed using the examination device. The numbers of cells before and after the culture were compared to evaluate the cell intake ratio at four tiers, A (120% or higher), B (80 to 119%), C (10 to 79%), and D (0 to 9%). The discrimination of the fluorescent cells was evaluated at four tiers based on the ratio of the fluorescent cells observed in the dark field image to the number of cells observed in the bright field image, A (60% or higher), B (30 to 59%), C (possible; 2 to 29%), and D (impossible; 0 to 1%). As for the presence/absence of the joint site, the surface of the stage after the sheet member was peeled off by a paper adhesive tape of an acrylic adhesive was observed by an electron microscope, and it was determined that a joint site was present if a part of the sheet member remained on the surface of the stage. The characteristics of the sheet members No. 5 to No. 23 and the evaluation results are shown in Table 2 below.

The thickness of the sheet member No. 2 stationarily fixed to the silicone case was observed using a contact-type film thickness gauge (a digimatic indicator ID-H manufactured by Mitsutoyo Corporation, having a flat terminal of Φ10), and the result was 6 μm. The ends of each of the sheet members No. 14 and No. 15 were observed using the digital microscope VHX5000 manufactured by Keyence Corporation at magnification ×250, and a three-dimensional image was obtained. As a result of measuring the step difference between the CMOS sensor and the sheet-member flat portion, the thickness of the sheet members was 27 μm and 20 μm, respectively. The surface of each of the sheet members No. 21 and No. 22 was observed using the digital microscope VHX5000 manufactured by Keyence Corporation at magnification ×1000, and a three-dimensional image was obtained. As a result of calculating a maximum height Sz from the CMOS sensor by the digital microscope VHX6000 manufactured by Keyence Corporation, the thickness of the sheet members was 9 μm and 5 μm, respectively.

TABLE 2

| | | Sheet | Average | Ratio of | Arithmetic | Maximum | | Intake | |
|---|---|---|---|---|---|---|---|---|---|
| | | width | diameter of | thick fibers | average height | height Sz | Joint | of | Discrimination of |
| No. | Stage | [mm] | fibers [μm] | [%] | Sa [μm] | [μm] | site | cells | fluorescent cells |
| 5 | Silicone case | 8 | 0.1 | 0 | 0.1 | 1 | None | B | A |
| 6 | Silicone case | 8 | 0.6 | 0 | 0.2 | 2 | None | B | A |
| 7 | Silicone case | 8 | 6.2 | 38 | 1.0 | 9 | None | B | A |
| 8 | Glass substrate | 5 | 1.1 | 0 | 0.2 | 2 | None | A | A |
| 9 | Glass substrate | 5 | 1.4 | 0 | 0.4 | 3 | None | A | A |
| 10 | Glass substrate | 5 | 2.5 | 0 | 1.0 | 9 | Present | B | A |
| 11 | Glass substrate | 5 | 3.3 | 12 | 1.6 | 14 | Present | B | A |
| 12 | Glass substrate | 5 | 5.4 | 24 | 0.5 | 8 | Present | B | A |
| 13 | Glass substrate | 5 | 6.4 | 57 | 0.4 | 10 | Present | B | A |
| 14 | CMOS sensor | 4 | 1.6 | 0 | 0.7 | 6 | None | A | C |
| 15 | CMOS sensor | 4 | 1.5 | 0 | 0.8 | 7 | None | A | C |
| 16 | CMOS sensor | 4 | 4.2 | 1 | 3.5 | 35 | Present | B | C |
| 17 | CMOS sensor | 4 | 3.5 | 2 | 4.4 | 58 | Present | B | B |
| 18 | CMOS sensor | 4 | 3.7 | 5 | 1.1 | 10 | Present | B | A |
| 19 | CMOS sensor | 4 | 3.9 | 8 | 3.3 | 29 | Present | B | B |
| 20 | CMOS sensor | 4 | 5.0 | 15 | 1.9 | 23 | Present | B | A |
| 21 | CMOS sensor | 4 | 4.6 | 25 | 1.1 | 9 | Present | B | A |
| 22 | CMOS sensor | 3 | 4.2 | 27 | 0.4 | 5 | Present | B | A |
| 23 | CMOS sensor | 3 | 6.6 | 50 | 0.8 | 6 | Present | B | B |

According to the results shown in Table 2, if at least one of the following conditions (a) to (d) is satisfied in the sheet member, the cell intake ratio can be 80% or higher and the discrimination of the luminescent cells is possible: (a) the width of the sheet member is 90 mm or less, and the height is 150 μm or less; (b) an average diameter of the fibers constituting the sheet member is in the range of 0.05 μm to and 10 μm; (c) the ratio of the fibers having the width of 6 μm or greater is in the range of 1% to 70%; or (d) the surface roughness of the sheet member is an arithmetic average height Sa in the range of 0.1 μm to 5 μm, and a maximum height Sz in the range of 1 μm to 90 μm. From the comparison of the sheets Nos. 5, 6, 8-10, 14 and 15 with the sheets Nos. 7, 11-13, and 16-23, it can be understood that the binding between the sheet member and the surface of the detection unit is encouraged if the sheet member includes fibers having the width of 6 μm or greater.

### Example 4

It was tested whether the sheets, for example the above-described sheet member 2b and sheet pieces 2b1, adsorb a substrate (luminescent substrate). In the test, as a detection unit, a plate reader (lumino meter) was used, and a reaction field was formed in a case formed on the plate reader. As cells, MCF7 was used, and liposome-encapsulated plasmid used for detecting light emission was charged into the cells. When one hour elapsed after the plasmid was charged into the cells, the cells were placed in the reaction field in the case. Herein, in the reaction field, no sheet member was spread on the bottom surface of the case, meaning the cells were directly placed on the bottom surface of the case; in other words, directly placed on the plate reader. In the reaction field, the placed cells were soaked in a culture solution, and the cells were cultured. In the test, the cells were seeded in the reaction field in the above-described manner when one hour elapsed, after the plasmid was charged into the cells. Then, after seeding, the cells were cultured in the reaction field for 24 hours.

In the test, after culturing the cells in the reaction field for 24 hours, a solution in which a substrate (luminescent substrate) dissolves was dropped onto (added to) the reaction field under each of the conditions X1 to X3 so as to cause light emission in the reaction field. As a substrate, a transient luminescent substrate was used. Then, in the plate reader, the emitted light was received, and the optical characteristics of the emitted light were detected. In the test, the amount of photons received by the plate reader within 60 seconds from the dropping time of the solution in which the substrate dissolves was detected as light emission intensity. Accordingly, the time during which the plate reader receives light in one session of detection (namely, exposure time) was 60 seconds.

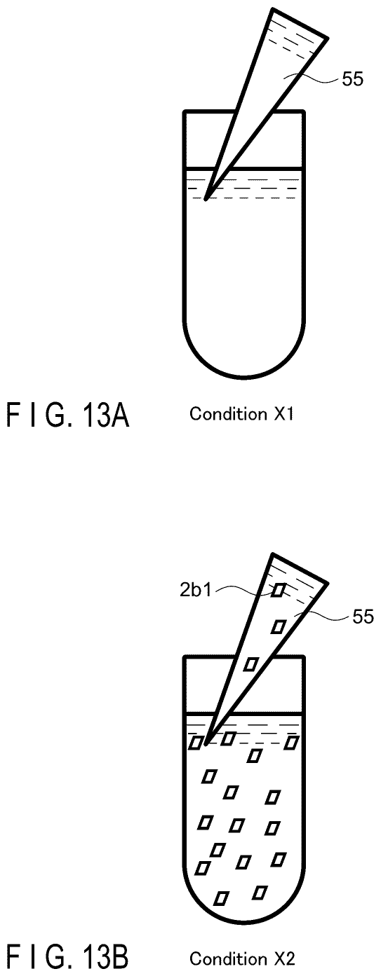

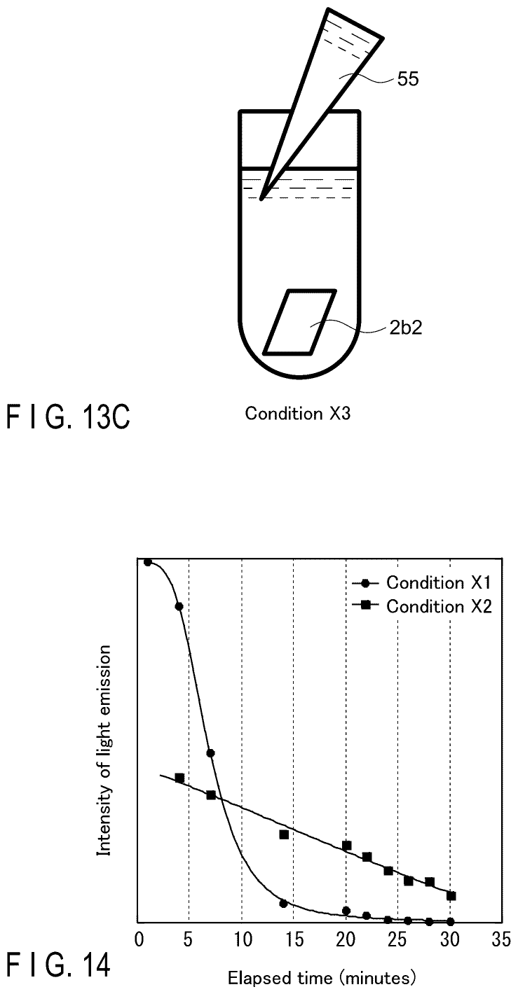

FIG. 13A shows the solution dropped onto the reaction field under the condition X1; FIG. 13B shows the solution dropped onto the reaction field under the condition X2; and FIG. 13C shows the solution dropped onto the reaction field under the condition X3. As shown in FIG. 13A, under the condition X1, the sheets such as sheet pieces 2b1 were not charged into the solution in which the substrate 55 dissolves. A part of the solution was taken out and dropped onto the reaction field. For this reason, the sheet was not charged into the reaction field under the condition X1.

Under the condition X2, a number of sheet pieces 2b1 were charged into the solution in which the substrate 55 dissolves. The sheet pieces 2b1 were formed by minutely dividing the sheet member 2b, as explained in connection with the example of FIG. 10. The sheet pieces 2b1 were formed as a single film of fibers having an average diameter of 3 μm. Furthermore, only after a number of sheet pieces 2b1 were charged into the solution and the sheet pieces 2b1 were dispersed or stirred into the solution to some extent, a part of the solution was taken out and dropped onto the reaction field. Under the condition X2, since the solution was dropped onto the reaction field as described above, a number of the sheet pieces 2b1 were dispersed in the solution dropped onto the reaction field, and a number of sheet members 2b1 (sheets) were charged into the reaction field together with the substrate (luminescent substrate).

Under the condition X3, a single sheet piece 2b2 was charged into the solution in which the substrate 55 dissolves. The sheet piece 2b2 was formed larger than each of the sheet pieces 2b1 used under the condition X2. The sheet piece 2b2 was formed as a single film of the fibers having an average diameter of 3 μm, similarly to the sheet piece 2b1. Only after a certain period of time elapsed since a single sheet piece 2b2 had been charged into the solution, a supernatant fluid that did not contain the sheet piece 2b2 was taken out of the solution. Then, the removed supernatant solution was dropped onto the reaction field. Since the supernatant fluid (solution) was dropped onto the reaction field as described above, no sheet, such as the sheet piece 2b2, was charged into the reaction field under the condition X3.

In the test, the solution in which the substrate 55 dissolves was dropped respectively onto the reaction fields under the conditions X1 and X2 at almost the same time, and the amount of photons received by the plate reader within 60 seconds from the solution dropping time was detected as light emission intensity for each of the conditions X1 and X2. The result shows that the light emission intensity under the condition X2 was 71.2%, compared to the light emission intensity under condition X1. Accordingly, it was demonstrated that a part of the substrate 55 adsorbed by the sheet piece 2b1 in the solution and the reaction between the substrate 55 adsorbed by the sheet piece 2b1 and the luciferase expressed in the cells was suppressed under the condition X2.

In the test, the solution in which the substrate 55 dissolves was dropped respectively onto the reaction fields under the conditions X1 and X3 at almost the same time, and the amount of photons received by the plate reader within 60 seconds from the solution dropping time was detected as light emission for each of the conditions X1 and X3. The result shows that the light emission intensity under the condition X3 is 69.5% compared to the light emission intensity under the condition X1. Accordingly, it was demonstrated that a part of the substrate 55 was adsorbed by the sheet piece 2b2 under the condition X3 during the time from when the sheet piece 2b2 was charged into the solution in which the substrate 55 dissolves to when the supernatant fluid was dropped onto the reaction field.

### Example 5

It was tested whether the sheets, for example the above-described sheet member 2b and sheet pieces 2b1, gradually release an adsorbed substrate (luminescent substrate). In the test, a reaction field was formed on the plate reader, in similar manner to the test conducted in Example 4. Similarly to the test in Example 4, MCF7 was used as cells, and liposome-encapsulated plasmid, which is used for detecting light emission, was charged into the cells. Similarly to the test in Example 4, the cells were seeded in the reaction field when one hour elapsed after the plasmid was charged into the cells, and the seeded cells were cultured in the reaction field for 24 hours. Then, after culturing the cells in the reaction field for 24 hours, a solution in which a substrate (luminescent substrate) dissolves was dropped onto (added to) the reaction fields respectively under the conditions X1 to X2 as described in the test of Example 4, so as to cause light emission in the reaction fields respectively. As a substrate, a transient luminescent substrate was used, similarly to the test in Example 4. Then, in the plate reader, the emitted light was received, and the optical characteristics of the emitted light were detected.

In the test, the detection was conducted at ten points in time within the 30 minutes elapsed since the dropping of the solution in which the substrate dissolves, under the conditions X1 and X2 respectively. Thus, the light emission in the reaction field was continuously observed for 30 minutes from the dropping of the solution on the reaction field. In the detection at each of the ten points in time in the 30 minutes from the dropping of the solution, an amount of photons received by the plate reader in 60 seconds was detected as the light emission intensity. Accordingly, a period of time during which the plate reader receives light in one session of detection (namely, exposure time) was 60 seconds.

FIG. 14 shows the change with time of light emission intensity in the test under each of the conditions X1 and X2. In FIG. 14, the abscissa axis shows a time elapsed from the dropping of the solution on the reaction field, and the ordinate axis shows light emission intensity. FIG. 14 shows the detection values at ten points in time when a detection was conducted by the data points for each of the conditions X1 and X2, and an approximate line that passes those ten data points or the proximity thereof. As shown in FIG. 14, under the condition X1, the light emission intensity was high immediately after the solution was dropped, but rapidly decreased when 5 minutes elapsed from the dropping of the solution. Then, when 15 minutes elapsed from the dropping of the solution, light emission scarcely occurred in the reaction field.

Under the condition X2 on the other hand, the light emission intensity immediately after the dropping of the solution was lower than that under the condition X1. However, under the condition X2, the light emission intensity gradually declines until 30 minutes elapsed from the dropping of the solution. For this reason, under the condition X2, the light emission occurred in the reaction field even after almost 30 minutes had elapsed from the dropping of the solution, the light emission intensity is maintained high to some extent.

Thus, it was demonstrated that, under the condition X2, a part of the substrate in the solution is adsorbed by the sheet piece 2b1, and the substrate adsorbed by the sheet piece 2b1 was gradually released to the reaction field. In other words, it was demonstrated that the substrate adsorbed by the sheet piece 2b1 is gradually released to the reaction field over a long period of time.

The examination device of at least one of the foregoing embodiments or examples includes a detection unit, a container arranged above the detection unit and made of a material having light-transmitting properties, and a sheet member arranged in the container. Thus, an examination device capable of culturing specimen cells at a high intake ratio, even cells difficult to culture outside of a body, and visualizing the activity of the living cells in a real-time manner.

In the examination device of at least one of the foregoing embodiment or examples, a reagent reacts with a measurement target and thereby causes light emission. The sheet can adsorb a reagent and gradually release the adsorbed reagent. It is thereby possible to provide an examination device that detects optical characteristics at high sensitivity in a detection unit.

A manufacturing method of an examination device, a cell detection method and an examination method in the foregoing embodiment and the like will be added as follows.

### APPENDIX 1

A manufacturing method of an examination device, the examination device comprising a detection unit, a case arranged above the detection unit and made of a material having light-transmitting properties, and a sheet member placed in the case, the method comprising:
directly forming the sheet member in the container by an electrospinning method.

### APPENDIX 2

A cell detection method which uses an examination device comprising a detection unit, a case arranged above the detection unit and made of a material having light-transmitting properties, and a sheet member placed in the case, the method comprising:
culturing a group of specimen cells in the case;
bringing a reagent capable of visualizing characteristics of the group of specimen cells as optical characteristics into contact with the group of specimen cells;
obtaining the optical characteristics by the detection unit; and
distinguishing target cells included in the group of specimen cells based on the optical characteristics.

### APPENDIX 3

The cell detection method according to Appendix 2, wherein
the optical characteristics are an amount of change in an amount of received light or an amount of change in wavelength when fluorescent light or visible light passes through the group of specimen cells.

### APPENDIX 4

The cell detection method according to Appendix 3, wherein
the optical characteristics are an amount of change in an amount of received light emitted from the group of specimen cells in a state where no external light is irradiated.

### APPENDIX 5

The cell detection method according to Appendix 2, wherein
the reagent includes at least one of a molecule that recognizes a biomolecule, protein, antibody, enzyme, nucleic acid, vector DNA, a stain for protein, or a stain for DNA.

### APPENDIX 6

The cell detection method according to Appendix 2, wherein
the reagent is encapsulated by one of a tissue-derived molecule, a biocompatible molecule, or a biodegradable molecule.

### APPENDIX 7

The cell detection method according to Appendix 6, wherein
the capsule includes a lipid molecule or polymer.

### APPENDIX 8

An examination method comprising:
causing light emission by a reaction between a reagent and a measurement target in a reaction field in which a sheet capable of adsorbing and gradually releasing the reagent is placed; and
receiving light emitted in the reaction field by a detection unit arranged near the reaction field, and detecting optical characteristics of the received light emitted in the reaction field.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An examination method comprising:
dispersing a plurality of pieces in a solution in which a reagent dissolves, the pieces being capable of adsorbing the reagent and gradually releasing the adsorbed reagent;
producing a measurement target in a reaction field or dropping the measurement target onto the reaction field, the reaction field being formed inside a container;
dropping the solution of the reagent onto the reaction field and thereby causing light emission by a reaction between the measurement target and the reagent; and
receiving light emitted in the reaction field by a detection unit, and detecting optical characteristics of the received light emitted in the reaction field, the container being arranged on the detection unit and the detection unit facing the reaction field.

2. The examination method according to claim 1, further comprising:
providing, by the detection unit, an accumulation value of light emission intensity during a predetermined time of light emission in the reaction field.

3. The examination method according to claim 2, wherein the predetermined time is between 3 seconds and 60 minutes.

4. The examination method according to claim 1, wherein
each of the plurality of pieces includes fibers of a biocompatible high polymer, and
an average diameter of the fibers is from 0.05 μm to 10 μm.

5. An examination method comprising:
causing interaction of a reagent and a measurement target in a presence of a plurality of porous sheet pieces in a reaction field of a container case, the plurality of porous sheet pieces having a random orientation of nonwoven fibers and being dispersed within the reaction field; and
detecting light emitted in the reaction field due to the interaction of the reagent and the measurement target in the presence of the plurality of porous sheet pieces by the detection unit, the detection unit being integrated into a bottom surface of the container case beneath the reaction field.

6. The examination method according to claim 5, further comprising:
providing, by the detection unit, an accumulation value of light emission intensity during a predetermined time of light emission in the reaction field.

7. The examination method according to claim 6, wherein the predetermined time is between 3 seconds and 60 minutes.

8. The examination method according to claim 5, wherein
each of the plurality of porous sheet pieces includes fibers of a biocompatible high polymer, and
an average diameter of the fibers is from 0.05 μm to 10 μm.
